(19) United States
(12) Patent Application Publication
(10) Pub. No.: US 2005/0283131 A1
(43) Pub. Date: Dec. 22, 2005

(54) Absorbent article having wings with non-bunching/twisting security

(75) Inventors: Teresa Marie Zander, Bonduel, WI (US); Angela Rae Heck, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/872,200

(22) Filed: Jun. 18, 2004

(51) Int. Cl. A61F 13/15

(52) U.S. Cl. 604/385.04

(57) ABSTRACT

An absorbent article (20) has a longitudinal-direction (22), a lateral cross-direction (24), an intermediate portion (76) interposed between a pair of longitudinally opposed end portions, and a pair of longitudinally-extending, laterally-opposed side edge regions. The article comprises a backsheet (28), a liquid-permeable topsheet (26) and an absorbent body (30) sandwiched between the backsheet (28) and the topsheet (26). At least one pair of wing-panels (42, 42) are operatively joined to the intermediate portion (76) of the article (20). Each wing-panel (42, 42) extends laterally from at least one corresponding lateral side edge region, and each wing-panel (42, 42) is configured to wrap about a wearer's undergarment. A panel fastener (44, 44a) can be joined to each wing-panel (42, 42), with each panel fastener (44, 44a) configured to operatively secure its corresponding wing-panel (42, 42) about the undergarment. In a particular aspect, an overall total, wing-panel area in a first lateral-half (60) of the article can be bilaterally asymmetric with respect to an overall total, wing-panel area in a second lateral-half (60a) of the article. In another aspect, each panel fastener (44, 44a) can be configured to attach to the undergarment of the wearer at an attachment location which has a fastener end-spacing (70). In desired arrangements, the fastener end-spacing can be not more than about 18% of an overall length (48) of the absorbent body (30).

### FIELD OF THE INVENTION

The present invention relates to a fastening system for an absorbent article. More particularly, the present invention pertains to a personal care article employed by adults, such as a feminine care pad, having a system of one or more wing-panels.

### BACKGROUND OF THE INVENTION

Absorbent products intended to absorb discharged body fluids are well known in the art. Such absorbent products generally comprise a fibrous mass or other absorbent body which can absorb and hold the body fluids. Similarly, it is well known that, feminine care articles have been employed to absorb and hold liquids, such as urine and/or menses. In particular arrangements, the feminine care articles have included wing portions which can help to hold the article in place at a selected location in a wearer's undergarment. In some arrangements, the wing portions have been integrally formed with one or more of the preexisting component layers that were employed to construct the article. In other arrangements, the wing portions have been separately provided components that are assembled and affixed to the final product. Various fasteners have been employed to secure the wing portions in a desired configuration during ordinary use. The fasteners have included adhesive fasteners as well as mechanical fasteners, and the mechanical fasteners have included conventional, hook-and-loop fasteners.

Conventional absorbent articles with wing portions, however, have not provided desired combinations of securement, comfort, performance and versatility. When conventional articles have been constructed with integrally formed wing components, it has been difficult to provide the wing portions with desired, differentiated characteristics. When conventional articles have been constructed with separately provided wing components, the finished product has not provided sufficient levels of aesthetic appeal and attractiveness. As a result, there has been a continued need for an improved article design that provides visual cues of desired functional benefits. Additionally, there has been a continued need for the versatility and enhanced performance that can arise from separately provided wing portions that are constructed from different materials, and configured to have selected characteristics and performance parameters.

### BRIEF DESCRIPTION OF THE INVENTION

Generally stated, an absorbent article has a longitudinal-direction, a lateral cross-direction, an intermediate portion interposed between a pair of longitudinally opposed end portions, and a pair of longitudinally-extending, laterally-opposed side edge regions. The article comprises a backsheet, a liquid-permeable topsheet and an absorbent body sandwiched between the backsheet and the topsheet. At least one pair of wing-panels are operatively joined to the intermediate portion of the article. Each wing-panel extends laterally from at least one corresponding lateral side edge region, and each wing-panel is configured to wrap about a wearer's undergarment. A panel fastener can be joined to each wing-panel, with each panel fastener configured to operatively secure its corresponding wing-panel about the undergarment. In a particular aspect, an overall total, wing-panel area in a first lateral-half of the article can be bilaterally asymmetric with respect to an overall total, wing-panel area in a second lateral-half of the article. In another aspect, each panel fastener can be configured to attach to the undergarment of the wearer at an attachment location which has a distinctive, fastener end-spacing. In particular configurations, the fastener end-spacing can be not more than about 18% of an overall length of the absorbent body.

By incorporating its various aspects, features and configurations, the article of the invention can more effectively maintain a desired positioning in a wearer's undergarment, and can be less susceptible to excessive shifting. As a result, the article can provide improved fit and/or comfort. Additionally, the article can help maintain a desired positioning relative to the wearer, can help provide a higher resistance to leakage, and can help increase the wear-time of an individual article.

### BRIEF DESCRIPTION OF THE DRAWINGS

The various features, aspects and advantages of the present invention will become better understood with reference to the following description, appended claims and accompanying drawings where:

FIG. 1 shows a partially cut away, plan view of a garment-side of a representative feminine care article where a pair of wing-panels are arranged in a laterally-closed position in which the wing-panels can wrap about a wearer's undergarment.

FIG. 2 shows a partially cut away, plan view of a bodyside of a representative feminine care article in which one wing-panel is arranged in a laterally-extended position.

FIG. 3 shows a bottom, plan view of a garment-side of a representative feminine care article in which one wing-panel is arranged in a laterally-extended position.

FIG. 4 shows a view of a representative longitudinal cross-section through a personal care article of the invention.

### DETAILED DESCRIPTION OF THE INVENTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

By the terms “particle,” “particles,” “particulate,” “particulates” and the like, it is meant that the adsorbent material is generally in the form of discrete units. The units can comprise granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

As used herein, the term “nonwoven” refers to a fabric web that has a structure of individual fibers or filaments which are interlaid, but not in an identifiable repeating manner.

As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

As used herein, the phrase “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

“Coform” as used herein is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

As used herein, the phrase “complex liquid” describes a liquid generally characterized as being a viscoelastic liquid comprising multiple components having inhomogeneous physical and/or chemical properties. It is the inhomogeneous properties of the multiple components that challenge the efficacy of an adsorbent material in the handling of complex liquids. In contrast with complex liquids, simple liquids, such as, for example, urine, physiological saline, water and the like, are generally characterized as being relatively low-viscosity and comprising one or more components having homogeneous physical and/or chemical properties. As a result of having homogeneous properties, the one or more components of simple liquids behave substantially similarly during absorption or adsorption.

Although a complex liquid is generally characterized herein as including specific components having inhomogeneous properties, each specific component of a complex liquid generally has homogeneous properties. Consider for example a representative complex body-liquid having three specific components: red blood cells, blood protein molecules and water molecules. Upon examination, one skilled in the art could easily distinguish between each of the three specific components according to their generally inhomogeneous properties. Moreover, when examining a particular specific component such as the red blood cell component, one skilled in the art could easily recognize the generally homogeneous properties of the red blood cells.

As used herein, the phrase “absorbent article” refers to devices which absorb and contain body liquids, and more specifically, refers to devices which are placed against or near the skin to absorb and contain the various liquids discharged from the body. The term “disposable” is used herein to describe absorbent articles that are not intended to be laundered or otherwise restored or reused as an absorbent article after a single use. Examples of such disposable absorbent articles include, but are not limited to: health care related products including surgical drapes, gowns, and sterile wraps; personal care absorbent products such as feminine hygiene products (e.g., sanitary napkins, pantiliners, tampons, interlabial devices and the like), infant diapers, children's training pants, adult incontinence products and the like; as well as absorbent wipes and covering mats.

Disposable absorbent articles such as, for example, many of the feminine care absorbent products, can include a liquid pervious topsheet, a substantially liquid impervious backsheet joined to the topsheet, and an absorbent core positioned and held between the topsheet and the backsheet. The topsheet is operatively permeable to the liquids that are intended to be held or stored by the absorbent article, and the backsheet may be substantially impermeable or otherwise operatively impermeable to the intended liquids. The absorbent article may also include other components, such as liquid wicking layers, liquid intake layers, liquid distribution layers, transfer layers, barrier layers, and the like, as well as combinations thereof. Disposable absorbent articles and the components thereof, can operate to provide a body-facing surface and a garment-facing surface. As used herein, “body-facing surface” means that surface of the article or component which is intended to be disposed toward or placed adjacent to the body of the wearer during ordinary use, while the “outward surface” or “outward-facing surface” is on the opposite side, and is intended to be disposed to face away from the wearer's body during ordinary use. The outward surface may be arranged to face toward or placed adjacent to the wearer's undergarments when the absorbent article is worn.

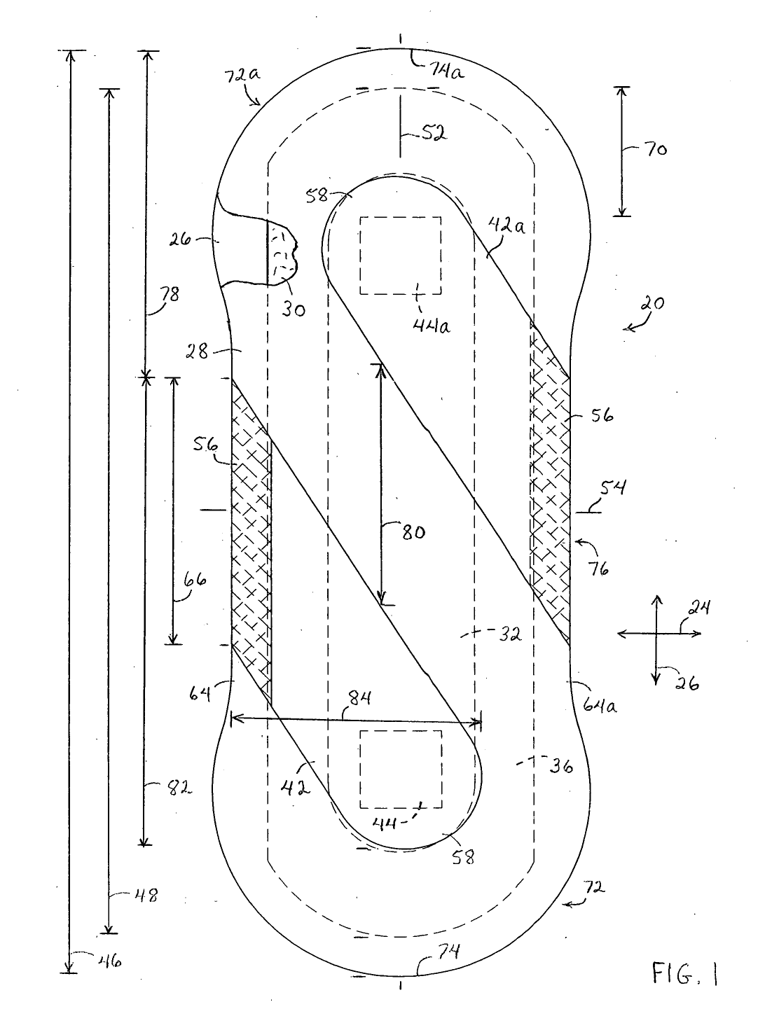

FIGS. 1 through 4 illustrate examples of a suitable article 20, such as the representatively shown personal care article, which is configured to incorporate the present invention. The personal care article can be a feminine care article, which can, for example, be a feminine care pad or napkin. The article has an appointed bodyside surface, an appointed garment-side surface, a lengthwise longitudinal-direction 22, a lateral cross-direction 24, a pair of longitudinally-extending, laterally-opposed side edge regions 64, and an intermediate portion 76 which is interposed between a pair of longitudinally opposed end portions 72 (72, 72a). The article also has a longitudinally extending centerline 52 and a laterally extending centerline 54. As representatively shown, the longitudinal dimension of the article is relatively larger than the lateral dimension of the article. In particular configurations, the article 20 can include a baffle or backsheet 28, a liquid-permeable cover or topsheet 26, and an absorbent body structure 30 which is operatively positioned and sandwiched between the backsheet 28 and topsheet 26. As representatively shown, peripheries of the topsheet and backsheet may be substantially entirely coterminous. Alternatively, the peripheries of the topsheet 26 and the backsheet 28 may be partially or entirely non-coterminous.

In a desired feature, the article can also include at least one pair of wing-panels 42. A first wing-panel 42 can be attached to a first side edge region 64 in the intermediate portion 76 of the article 20, and a second wing-panel 42a can be attached to a second side edge region 64a in the intermediate portion 76 of the article 20. Each wing-panel 42 (42, 42a) can be configured to wrap about an undergarment of a wearer. A panel fastener 44 can be joined to each wing-panel 42, and each panel fastener 44 can be configured to operatively secure its corresponding wing-panel 42 about the undergarment of the wearer during ordinary use. Each wing-panel 42 can include a panel base section 56 and a panel free-end region 58. Each panel base section 56 can be operatively joined to its corresponding side edge region 64 of the article 20. Each panel free-end region 58 can extend from its corresponding panel base section 56, and can be configured to operatively wrap around an appointed region of the wearer's undergarment. For example, the free-end regions 58 of the wing-panels can be wrapped about a crotch region of the wearer's undergarment. In a particular aspect, an overall total, wing-panel area in a first lateral-half 60 of the article can be configured to be significantly, bilaterally asymmetric with respect to an overall total wing-panel area in a second lateral-half 60a of the article 20. In another aspect, each panel fastener can be configured to operatively attach to the undergarment of the wearer at a distinctive attachment location during ordinary use. Desired configurations of the invention can provide arrangements in which each panel fastener 44 is configured to have a selected fastener end-spacing 70. In a particular feature, the fastener end-spacing 70 can be not more than a maximum of about 18% of an overall length 48 of the absorbent body 30. Further features of the invention are set forth in the present disclosure.

By incorporating its various aspects, features and configurations (alone or in desired combinations), the article of the invention can more effectively maintain a desired positioning in a wearer's undergarment, and can be less susceptible to excessive shifting. As a result, the article can provide improved fit and/or comfort, and can help provide improved stay-in-place security. Additionally, the article can help provide increased resistance to leakage and an increased wearing time of an individual article.

The cover or topsheet 26 may include any material that can be configured to provide the topsheet with an operative level of liquid-permeability. The topsheet may be constructed with one or more layers of suitable materials, and may be a composite material. For example, the topsheet layer can include a woven fabric, a nonwoven fabric, a polymer film, a film-fabric laminate or the like, as well as combinations thereof. Examples of a nonwoven fabric include, spunbond fabric, meltblown fabric, coform fabric, a carded web, a bonded-carded-web, a bicomponent spunbond fabric or the like as well as combinations thereof. For example, the topsheet layer can include a woven fabric, a nonwoven fabric, a polymeric film that has been configured to be operatively liquid-permeable, or the like, as well as combinations thereof. Other examples of suitable materials for constructing the topsheet layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable topsheet layer material can include a bonded-carded-web composed of polypropylene and polyethylene, such as has been used as a topsheet stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. In a desired arrangement, the topsheet layer 26 can be configured to be operatively liquid-permeable with regard to the liquids that the article is intended to absorb or otherwise handle. The operative liquid-permeability may, for example be provided by a plurality of pores, perforations, apertures or other openings, as well as combinations thereof, that are present or formed in the topsheet layer. The apertures or other openings can help increase the rate at which bodily liquids can move through the thickness of the topsheet layer and penetrate into the other components of the article (e.g. into the absorbent structure 30). The selected arrangement of liquid-permeability is desirably present at least on an operative portion of the topsheet layer that is appointed for placement on the body-side of the article. The topsheet layer 26 can provide comfort and conformability, and can function to direct bodily exudates away from the body and toward the absorbent structure 30. In a desired feature, the topsheet layer 26 can be configured to retain little or no liquid in its structure, and can be configured to provide a relatively comfortable and non-irritating surface next to the body-tissues of a female wearer. The topsheet layer 26 can be constructed of any material which is also easily penetrated by bodily fluids that contact the surface of the topsheet layer.

The topsheet 26 can also have at least a portion of its bodyside surface treated with a surfactant to render the topsheet more hydrophilic. The surfactant can permit arriving bodily liquids to more readily penetrate the topsheet layer. The surfactant may also diminish the likelihood that the arriving bodily fluids, such as menstrual fluid, will flow off the topsheet layer rather than penetrate through the topsheet layer into other components of the article (e.g. into the absorbent body structure). In a particular configuration, the surfactant can be substantially evenly distributed across at least a portion of the upper, bodyside surface of the topsheet 26 that overlays the upper, bodyside surface of the absorbent.

The topsheet 26 may be maintained in secured relation with the absorbent structure 30 by bonding all or a portion of the adjacent surfaces to one another. A variety of bonding techniques known to one of skill in the art may be utilized to achieve any such secured relation. Examples of such techniques include, but are not limited to, the application of adhesives in a variety of patterns between the two adjoining surfaces, entangling at least portions of the adjacent surface of the absorbent with portions of the adjacent surface of the topsheet, or fusing at least portions of the adjacent surface of the topsheet to portions of the adjacent surface of the absorbent.

The topsheet 26 typically extends over the upper, bodyside surface of the absorbent structure to provide a bodyside liner, but can alternatively extend around the article to partially or entirely, surround or enclose the absorbent structure. Alternatively, the topsheet 26 and the backsheet 28 can have peripheral margins which extend outwardly beyond the terminal, peripheral edges of the absorbent structure 30, and the extending margins can be joined together to partially or entirely, surround or enclose the absorbent structure.

The baffle or backsheet 28 may include a layer constructed of any operative material, and may or may not be configured to be liquid-permeable. In a particular configuration, the cover or backsheet 28 may be configured to provide an operatively liquid-impermeable layer. The backsheet may, for example, include a polymeric film, a woven fabric, a nonwoven fabric or the like, as well as combinations or composites thereof. For example, the backsheet may include a polymer film laminated to a woven or nonwoven fabric. In a particular feature, the polymer film can be composed of polyethylene, polypropylene, polyester or the like, as well as combinations thereof. Additionally, the polymer film may be micro-embossed. Desirably, the backsheet 28 can operatively permit a sufficient passage of air and moisture vapor out of the article, particularly out of an absorbent (e.g. storage or absorbent structure 30) while blocking the passage of bodily liquids. An example of a suitable backsheet material can include a breathable, microporous film, such as a HANJIN Breathable backsheet available from Hanjin Printing, Hanjin P&C Company Limited, a business having offices located in Sahvon-Ii.Jungan-mvu.Kongiu-City, Chung cheong nam-do, Republic of South Korea. The backsheet material is a breathable film, which is white in color, dimple embossed, and contains: 47.78% calcium carbonate, 2.22% TiO₂, and 50% polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable backsheet material can include a closed cell polyolefin foam. For example, a closed cell polyethylene foam may be employed. Still another example of a backsheet material would be a material that is similar to a polyethylene film which is used on commercially sold KOTEX brand pantiliners, and is obtainable from Pliant Corporation, a business having offices located in Schaumburg, Ill., USA.

The structure of the absorbent body 30 can be operatively configured to provide a desired level of absorbency or storage capacity. More particularly, the absorbent body can be configured to hold a liquid, such as urine, menses, other complex liquid or the like, as well as combinations thereof. As representatively shown, the absorbent body can include a matrix of absorbent fibers and/or absorbent particulate material, and the absorbent fiber can include natural and/or synthetic fiber.

The absorbent structure 30 may also include superabsorbent material. Superabsorbent materials suitable for use in the present invention are known to those skilled in the art, and may be in any operative form, such as particulate form. Generally stated, the water-swellable, generally water-insoluble, hydrogel-forming polymeric absorbent material (superabsorbent) is capable of absorbing at least about 10, desirably about 20, and possibly about 100 times or more its weight in water. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as The Dow Chemical Company, Hoechst Celanese Corporation, Allied Colloid Inc., and Stockhausen, Inc.

The absorbent body 30 can be substantially unitary with a non-uniform structure or a generally uniform structure. Alternatively, the absorbent body may include a composite structure having a selected plurality of strata or layers. For example, the absorbent body structure may include an intake layer, a distribution layer, a transfer layer, a transfer-delay layer, a shaping layer, a retention layer or the like, as well as combinations thereof. The various strata and/or layers may be stacked, distributed or otherwise arranged in any operative sequence or configuration.

Additionally, the absorbent article can include any desired pattern or array of embossments. In particular aspects, the embossments may be formed on the bodyside surface of the article. Desired arrangements can include an absorbent body structure that has embossment regions formed on at least its bodyside surface. Similarly, the other employed components of the article can also include corresponding embossed regions.

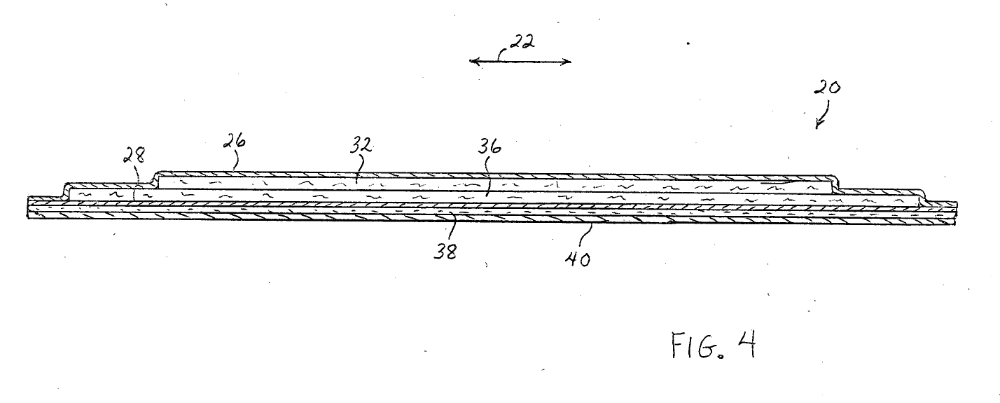

With reference to FIGS. 1 and 4, the absorbent composite may, for example, include either or both of an intake layer 32, and an absorbent retention layer 36. As representatively shown, the absorbent body can include an absorbent retention layer 36 which is positioned between the topsheet 26 and the backsheet 28. Additionally, the absorbent body can include an intake layer 32 which is positioned between topsheet 26 and the retention layer 36. The absorbent body can further include one or more additional layers positioned between the topsheet 26 and backsheet 28. The various individual layers may be separately provided layer-components, may be integrally formed together, or may be provided as any operative combination of separately-provided and integrally-formed layers.

The intake layer 32 can provide a desired intake of liquid and distribution of the liquid. The intake layer may include natural fibers (e.g. cellulose fibers), synthetic fibers, superabsorbent materials, a woven fabric; a nonwoven fabric; a wet-laid fibrous web; a substantially unbonded airlaid fibrous web; an operatively bonded, stabilized-airlaid fibrous web; a multifunctional stabilized-airlaid fibrous web; or the like, as well as combinations thereof.

In a particular arrangement, the intake layer can be a thermally-bonded, stabilized-airlaid fibrous web. The stabilized-airlaid web can have a basis weight of about 175-200 g/m², and a density of about 0.06-0.08 grams/cm³. Suitable stabilized-airlaid webs are available from Concert Fabrication, a business having offices located in Gatineaux, Quebec, Canada.

The retention layer 36 can provide a desired, absorbent retention or storage function, and may provide a selected shaping of the absorbent article. The retention layer may include natural fibers (e.g. cellulose fibers), synthetic fibers, superabsorbent materials, a woven fabric; a nonwoven fabric; a wet-laid fibrous web; a substantially unbonded airlaid fibrous web; an operatively bonded, stabilized-airlaid fibrous web; a multifunctional stabilized-airlaid fibrous web; or the like, as well as combinations thereof.

In desired configurations, the absorbent body 30 can provide an overall, total absorbent capacity which is at least a minimum of about 10 grams of menses simulant. The overall absorbent capacity can alternatively be at least about 20 grams of menses simulant, and can optionally be at least about 30 grams of menses simulant to provide improved performance. In other aspects, the overall absorbent capacity can be up to a maximum of about 120 grams of menses simulant, or more. The overall absorbent capacity can alternatively be up to about 100 grams of menses simulant, and can optionally be up to about 90 grams of menses simulant to provide improved effectiveness.

In a representative example, the article can include a nonwoven spunbond topsheet having a basis weight of about 20.3 g/m², and treated with 0.3% AHCOVEL surfactant. The AHCOVEL surfactant acts as a wetting agent to promote liquid intake. The intake layer of the article can comprise a generally homogeneous, stabilized-airlaid fibrous web having a basis weight of about 250 g/m², and a density of about 0.14 g/cm³. The transfer layer can include a rose colored 27.1 g/m² spunbond nonwoven fabric treated with 0.3% AHCOVEL surfactant. The transfer layer can help to regulate liquid transfer from the intake layer to the retention layer. The retention layer can be a generally homogeneous, stabilized-airlaid fibrous web having a density of about 0.08 g/cm³ and a basis weight of about 175 g/m². The backsheet can be a 0.7 mil (0.018 mm) thick, micro-embossed, polypropylene film, which can substantially prevent liquid from reaching the undergarment and can act as the substrate for the absorbent structure.

The article 20 can include a system of side “wings” or wing-panel portions 42 which are positioned along both lateral side regions 64 of the article. The wing-panels can be separately provided members that are subsequently attached or otherwise joined to the intermediate portion of the article 20. The separately provided wing-panel member can be operatively attached to at least one of the backsheet 28 and/or topsheet 26. In an alternative arrangement, each wing-panel 42 can be provided by an integrally-formed side portion of the article 20. The wing-panels may be integrally formed from another component of the article, such as the topsheet and/or the backsheet, and operatively connected or otherwise joined to appointed sections of the article side regions 64 along the intermediate portion of the article.

In a particular configuration, each wing-panel 42 can include an integrally-formed side portion of the backsheet 28 which extends laterally past a corresponding, terminal side edge of the absorbent body 30. In another arrangement, each wing-panel 42 can include an integrally-formed side portion of the topsheet 26 which extends laterally past a corresponding, terminal side edge of the absorbent body. Still a further arrangement can have a configuration wherein each wing-panel 42 includes a panel composite. The panel composite can, for example, include an integrally-formed side portion of the backsheet 28 which extends laterally past a corresponding, terminal side edge of the absorbent body, and an integrally-formed cooperating, side portion of the topsheet 26 which extends laterally past the corresponding, terminal side edge of the absorbent body. The cooperating, side portion of the topsheet 26 is positioned in facing relation with the integrally-formed side portion of the backsheet 28.

As representatively shown, the article 20 has a main article-chassis that holds the primary absorbent body 30. Where the wing-panels are separately provided members, a person of ordinary skill can readily discern the wing-panels from the main article-chassis. Additionally, the base-sections 56 of the wing-panels, and the intersect points where the wing-panels transition from the article-chassis can be readily determined.

Where the wing-panels are integrally formed from one or more other components of the article, the article typically has a side edge-contour which can be discontinuous or generally continuous along a transition from the article-chassis to an individual wing-panel 42. Where the transition is discontinuous, the transition point is readily discernable. Where the transition is continuous, the point of transition from a side-edge region of the article to a wing-panel of the article will, for the purposes of the present disclosure, lie on an inner-most portion of the transitioning edge-contour that is shaped to be concave-outward. Additionally, the transition point occurs at a point where a tangent line to the side edge-contour lies at a 45 degree angle, relative to the longitudinal centerline 52.

In the cooperating pair of wing-panels, the first wing-panel 42 can be attached or otherwise operatively joined to a first side edge region 64 in the intermediate portion 76 of the article 20, and the second wing-panel 42a can be attached to a second side edge region 64a in the intermediate portion of the article. Each wing-panel 42 (42, 42a) can be configured to operatively wrap about an undergarment of a wearer.

The wing-panels can have an appointed storage position in which the wing-panels 42 are directed generally inwardly toward the longitudinally-extending centerline 52. The wing-panel that is connected to extend from one side margin may have sufficient cross-directional length to extend and continue past the centerline 52 to approach the laterally opposite side margin of the article. The storage position of the wing-panels can ordinarily represent an arrangement observed when article is first removed from its wrapper or other packaging. Prior to placing the article into a bodyside of an undergarment prior to use, the wing-panels 42 can be selectively arranged to extend laterally from the side regions 64 of the article intermediate portion. After placing the article in the undergarment, the wing-panels 42 can be operatively wrapped and secured in an in-use position around the side edges of the undergarment crotch portion to help hold the article in place. Typically, the wing-panels are configured to secure the article to a crotch portion of the wearer's undergarment.

The wing-panel portions 42 can have any operative construction, and can include a layer of any operative material. Additionally, each wing-panel can comprise a laminate or other composite material. For example, the wing-panels may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web or the like, as well as combinations thereof.

The wing-panel materials can be substantially non-stretchable or may be stretchable. The wing-panel material may also be capable of providing a selected amount of elastomeric stretch and retraction. In particular configurations, the wing-panel material can exhibit a maximum stretch elongation value of up to about 300%, or more. In other configurations, the wing-panel material can exhibit a minimum stretch elongation value of 50%. In other features, the wing-panel material can have a basis weight which is within the range of about 0.5-3 ounces per square yard (about 17-102 g/m²). By employing such wing-panel materials, the wing-panel can help provide desired fit characteristics, and can help provide improved leak protection.

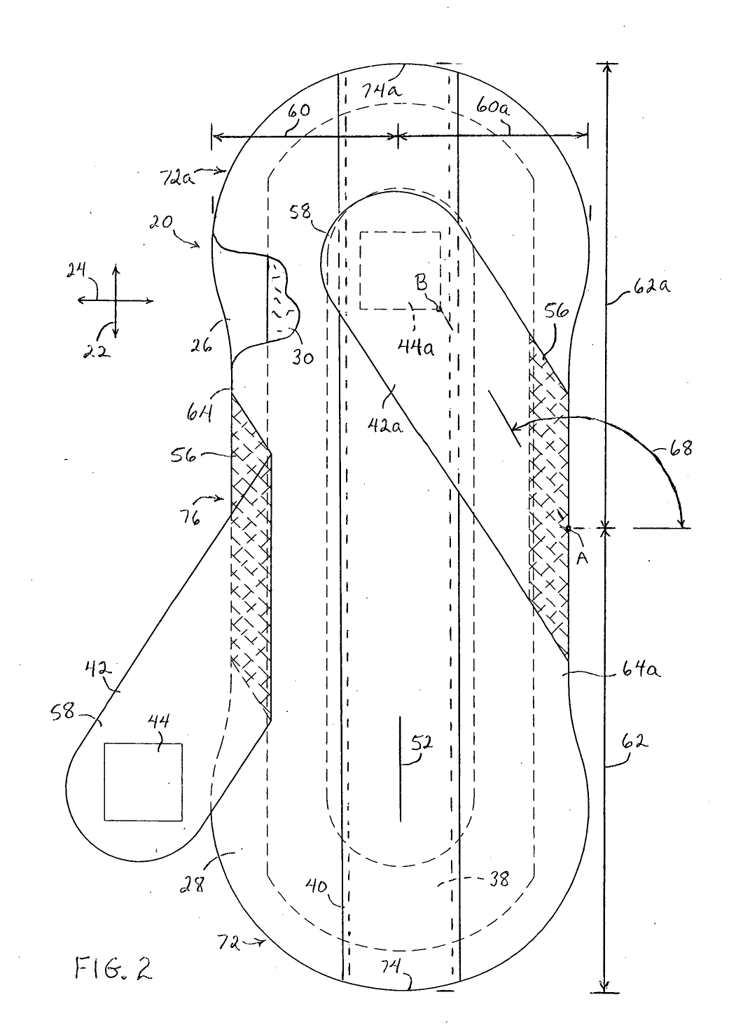

With reference to FIGS. 1 and 2, the wing-panel 42 can have a lateral, transverse-extent 84 and a lengthwise, longitudinal-extent 82. In desired configurations, a maximum longitudinal-extent 82 of the wing panel 42 can be not more than about 60% of a total, overall longitudinal length 48 of the absorbent body 30. The longitudinal-extent 82 of each wing-panel can alternatively be not more than about 55%, and can optionally be not more than about 53% of the overall longitudinal length of the absorbent body to provide desired benefits.

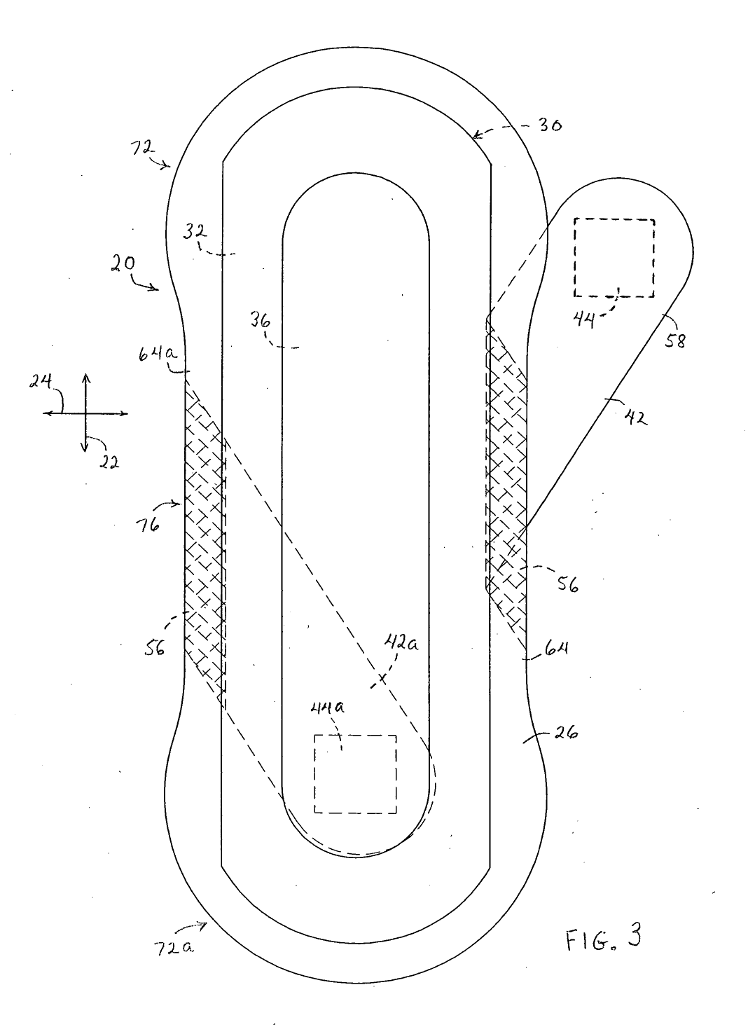

As representatively shown in FIGS. 2 and 3, the wing-panel 42, can be configured to extend laterally outwardly from the transversely-opposed side edge regions of the article. The pair of cooperating wing-panels are transversely-opposed but are significantly non-aligned along the cross-direction 24. The pair of wing-panels 42, 42a can have approximately the same area-size, and can have approximately the same shape. To a significant extent, however, the pair of wing-panels are not configured to be mirror-images of each other, particularly with respect to the longitudinally centerline 52.

Each wing-panel 42 can be positioned in an open position with its distal, movable end extended away from the longitudinal centerline 52 of the article. When all of the wing-panels (42, 42a) are positioned in their open position, at least a significant majority of a total area of all of the wing-panel (or wing-panels) located in a first lateral-half 60 of the article 20 can be located in a first longitudinal half-length 62 of the article. In particular aspects, a minimum of about 80% of the overall total wing-panel area located in a first lateral-half 60 of the article 20 can be located in the first longitudinal half-length 62 of the article. Alternatively at least about 85%, and optionally at least about 90% of the overall total wing-panel area located in the first lateral-half 60 of the article 20 can be located in the first longitudinal half-length 60 of the article to provide desired benefits. Further aspects can have about 95%, or more of the overall total wing-panel area in the first lateral-half 60 of the article located in the first longitudinal half-length 62 of the article.

A further feature can have a configuration in which a significant majority of a total area of all of the wing-panel (or wing-panels) located in a second lateral-half 60a of the article 20 is located in a second longitudinal half-length 62a of the article, when all of the wing-panels (42, 42a) are extended in their open position. In particular arrangements, at least about 80% of the total wing-panel area located in the second lateral-half 60a of the article 20 can be located in the second longitudinal half-length 62a of the article. Alternatively at least about 85%, and optionally at least about 90% of the overall total wing-panel area positioned in the second lateral-half 60a of the article can be located in the second longitudinal half-length 62a of the article. Further aspects can have about 95%, or more of the overall total wing-panel area in the second lateral-half 60a of the article located in the second longitudinal half-length 62a of the article.

If the distributions of the wing-panel areas are outside the desired values, the article can exhibit excessive bunching, twisting or shifting of the article when the article is worn in an undergarment. As a result, the article can be uncomfortable and be susceptible to excessive leakage.

Each wing-panel 42 can include a panel-fastener component 44 which is operatively joined to a major facing surface of the associated wing-panel. Such major surface is typically appointed to engage and become operatively attached to an outward-facing surface of the wearer's undergarment. The panel-fastener 44 can include any operative fastener component, such as a component of an interengaging mechanical faster, an adhesive fastener, a cohesive fastener, a magnetic fastener, an electromechanical fastener or the like, as well as combinations thereof. The mechanical fastener component may, for example, include a suitable hook component, such as a J-hook, mushroom-head hook, flat-top nail-head hook, a palm-tree hook, a multiple-J hook or the like, as well as combinations thereof. Optionally, the mechanical fastener component may, for example, include a suitable loop component. The loop component may, for example, include a knit fabric, a woven fabric, a nonwoven fabric or the like, as well as combinations thereof.

When all of the wing-panels (42, 42a) are extended in their open position, a significant majority of a total area of all of the panel fastener (or fasteners) positioned in a first lateral-half 60 of the article 20 can be located in a first longitudinal half-length 62 of the article. Particular aspects of the invention can have a configuration in which at least about 80% of a total area of all of the panel fastener or fasteners (44) positioned in the first lateral-half 60 of the article 20 can be located in a first longitudinal half-length 62 of the article. Alternatively, at least about 90% of the total area of all of the panel fastener or fasteners (44) positioned in the first lateral-half 60 of the article 20 can be located in the first longitudinal half-length 62 of the article, and optionally, about 100% of the total area of all of the panel fastener or fasteners (44) positioned in the first lateral-half 60 of the article 20 can be located in the first longitudinal half-length 62 of the article to provide desired levels of effectiveness.

In another feature, when all of the wing-panels (42, 42a) are extended in their open position, a significant majority of a total area of all of the panel fastener (or fasteners) positioned in a second lateral-half 60a of the article 20 can be located in a second longitudinal half-length 62a of the article. Particular arrangements can have a configuration in which at least about 80% of the total area of all of the panel fastener (or fasteners) positioned in the second lateral-half 60a of the article 20 can be located in a second longitudinal half-length 62a of the article. Alternatively, at least about 90% of the overall area of the second panel fastener or fasteners (44a) can be located in the second longitudinal half-length 62a of the article, and optionally, about 100% of the overall area of the second panel fastener 44a can be located in the second longitudinal half-length 62a to provide desired benefits.

Each wing-panel 42 can include a panel base section 56 and a panel free-end region 58. Each panel base section 56 can be operatively joined to its corresponding side edge region 64 of the article 20, and each panel free-end region 58 can extend from its corresponding panel base section 56. The free-end region can be further configured to operatively wrap around an appointed region of the wearer's undergarment. For example, the free-end regions 58 of the wing-panels can be wrapped about a crotch region of the wearer's undergarment.

Each wing panel 42 can have a longitudinally-extending base-length 66. In particular aspects, the panel base-length can be at least a minimum of about 25% of the overall length 48 of the absorbent body 30. The panel base-length can alternatively be at least about 28%, and can optionally be at least about 31% of the overall length of the absorbent body to provide desired benefits. In other aspects, the panel base-length can be up to a maximum of about 42%, or more, of the overall length 48 of the absorbent body 30. The panel base-length can alternatively be up to about 40%, and can optionally be up to about 38% of the overall length 48 of the absorbent body 30 to provide desired levels of effectiveness.

In another aspect, the base-length 66 of each panel base section can be at least a minimum of about 6 cm. The panel base-length can alternatively be at least about 6.5 cm, and can optionally be at least about 7.5 cm to provide desired benefits. In other aspects, the panel base-length 66 can be up to a maximum of about 10 cm, or more. The panel base-length can alternatively be up to about 9 cm, and can optionally be up to about 8.5 cm to provide desired levels of effectiveness.

If the panel base-length 66 is outside the desired values, the article can exhibit an increased bunching, twisting and/or shifting of the article while the article is in the wearers undergarment. As a result, the article can undesirably provide reduced comfort and reduced resistance to leakage.

With reference to FIG. 1, the article 20 includes first and second, longitudinally opposed, article end-edges 74 (74, 74a). In a desired feature, the panel base section 56 of each wing panel 42 can be longitudinally spaced from each article end-edge 74 by a distinctive base spacing-distance 78. In particular aspects, the base spacing-distance can be at least a minimum of about 25% of the overall length 48 of the absorbent body 30. The base spacing-distance can alternatively be at least about 30%, and can optionally be at least about 32% of the overall length of the absorbent body to provide improved benefits. In other aspects, the base spacing-distance can be up to a maximum of about 50% of the overall length 48 of the absorbent body 30, or more. The base spacing-distance can alternatively be up to about 40%, and can optionally be up to about 36% of the overall length of the absorbent body to provide improved effectiveness.

If the base spacing-distance 78 is outside the desired values, the article can exhibit an increased bunching, twisting and/or shifting of the article while the article is in the wearer's undergarment. As a result, the article can undesirably provide reduced comfort and reduced resistance to leakage.

With reference to FIG. 2, an overall, total area of the incorporated array of wing-panel (or wing-panels) employed in the first lateral-half 60 of the article can be configured to be bilaterally asymmetric with respect to an overall, total area of the array of wing-panels employed in the second lateral-half 60a of the article 20. The bilateral asymmetry of the areas provided by the employed arrays of wing-panels is present in a significant amount. The bilateral asymmetry is significantly more than minor imperfections and differences arising from ordinarily accepted manufacturing tolerances.

The at least one pair of wing-panels 42, 42a can be contoured and shaped to be longitudinally asymmetric with respect to each other. In a particular feature, a first free-end region 58 of the first wing-panel 42 can significantly extend toward a first end portion 72 of the article 20, and a second free-end region 58a of the second wing-panel 42a can significantly extend toward a longitudinally opposite, second end portion 72a of the article. In other features, each of the wing-panels in the cooperating pair can have an approximately identical or a substantially matching size and shape, and the first wing-panel can have a configuration and positioning which is effectively rotated relative to the second wing-panel by a rotational angle of approximately 180 degrees (e.g. FIGS. 1 and 2).

As representatively shown, the article 20 can have an overall article shape that is bilaterally asymmetric, as observed along the transverse cross-direction 24 with respect to the longitudinally extending centerline 52 of the article. The bilateral asymmetry is present in a significant amount, and is significantly more than minor imperfections and differences arising from ordinary, accepted manufacturing tolerances.

Each panel fastener 44 can be configured to have a fastener end-spacing 70. As representatively shown in FIG. 1, the end-spacing 70 is a distance between a terminal, longitudinal-end edge of the absorbent body 30, and a most proximate, terminal, longitudinal-end edge of the closest panel-fastener 44.

In particular aspects, the fastener end-spacing 70 can be as low as about 0% of the overall length 48 of the absorbent body 30. The fastener end-spacing 70 can alternatively be at least about 3%, and can optionally be at least about 6% of the overall length of the absorbent body to provide desired benefits. In other aspects, the fastener end-spacing can be up to a maximum of about 18% of the overall length 48 of the absorbent body 30, or more. The fastener end-spacing 70 can alternatively be up to about 15%, and can optionally be up to about 11% of the overall, absorbent body length 48 to provide desired levels of effectiveness.

In another aspect, each panel fastener 44 can be configured to have a fastener end-spacing 70 which can be as low as about 0 cm. The fastener end-spacing can alternatively be at least about 0.65 cm, and can optionally be at least about 1.5 cm to provide desired benefits. In other aspects, the fastener end-spacing can be up to a maximum of about 4 cm, or more. The fastener end-spacing can alternatively be up to about 3.3 cm, and can optionally be up to about 2.5 cm to provide desired effectiveness.

If the fastener end-spacing 70 is outside the desired values, the article can exhibit an increased bunching, twisting and/or shifting of the article while the article is in the wearer's undergarment. As a result, the article can undesirably provide reduced comfort and reduced resistance to leakage.

With reference to FIG. 2, each wing-panel 42 and its corresponding panel fastener 44 can be configured to provide a fastener angle 68. The fastener angle 68 is the angle between a line parallel to the longitudinal centerline 52, and a line that extends from a point A to a point B when the wing-panel is in the ordinary in-use position. Point A is the center of the panel base length 66, as determined along the terminal side edge of the article. Point B is a point on the panel fastener 44 that is relatively closest to Point A.

In particular aspects, the fastener angle 68 can be at least a minimum of about 110 degrees. The fastener angle 68 can alternatively be at least about 120 degrees, and can optionally be at least about 135 degrees to provide desired benefits. In other aspects, the fastener angle 68 can be up to a maximum of about 160 degrees, or more. The fastener angle 68 can alternatively be up to about 150 degrees, and can optionally be up to about 140 degrees to provide desired effectiveness.

If the fastener angle 68 is outside the desired values, the panel fastener can poorly situated when the associated wing-panel 42 is in its in-use position. The article can then exhibit an increased bunching, twisting and/or shifting of the article while the article is disposed in the wearer's undergarment. As a result, the article can undesirably provide reduced comfort and reduced resistance to leakage.

With reference to FIG. 1, the at least one pair of wing-panels 42, 42a can be configured to substantially avoid overlapping each other when the wing-panels 42 (42, 42a) are in an in-use position, as wrapped or otherwise operatively positioned about the wearer's undergarment during ordinary use. In another feature, the at least one pair of wing-panels 42, 42a can be configured to provide a minimum inter-panel gap 80 between the wing-panels when the wing-panels are in the ordinary in-use position. As representatively shown, the inter-panel gap distance is measured along a line parallel to the longitudinal direction 22.

In particular aspects, the minimum inter-panel gap 80 can be not less than about 0.5 cm. The minimum inter-panel gap 80 can alternatively be not less than about 2 cm, and can optionally be not less than about 6 cm to provide desired benefits. In other aspects, the minimum inter-panel gap 80 can be up to about 7.5 cm, or more. The inter-panel gap 80 can alternatively be up to about 7 cm, and can optionally be up to about 6.5 cm to provide improved desired effectiveness.

If the inter-panel gap 80 is outside the desired values, the article can provide a less efficient use of material, and can exhibit undesired levels of bunching, twisting and/or shifting while the article is disposed in the wearer's undergarment. As a result, the article can undesirably provide greater cost, reduced comfort and reduced resistance to leakage.

With reference to FIGS. 2 and 4, a selected configuration of a garment-attachment mechanism 38 may be operatively distributed and joined onto the garment-side surface of the article 20 to help secure the article to the undergarment. The garment-fastener can include any operative fastener mechanism, such as a component of an interengaging mechanical fastener, an adhesive fastener, a cohesive fastener, a magnetic fastener, and electromechanical fastener or the like, as well as combinations thereof. For example, the garment-fastener can be provided by the representatively shown adhesive 38, and the garment-fastener may be arranged any operative pattern, such as one or more strip regions that are distributed onto the garment-side of the article. Typically, an operative garment adhesive can be distributed over the garment-side of the backsheet, and one or more layers or sheets of release material 40 can be removably placed over the garment adhesive during storage prior to use.

In a desired arrangement, at least a significant portion of the garment-attachment mechanism 38 can be located along the longitudinal centerline 52 of the article. A further feature can include an arrangement in which each panel fastener 44 and a cooperating portion of the garment-attachment mechanism 38 are configured to operatively sandwich a portion of the undergarment between such panel fastener 44 and its corresponding portion of the garment-attachment mechanism 38. The sandwiched arrangement would occur when each wing-panel 42 is in its in-use position, as would be observed when the wing-panels are wrapped or otherwise operatively positioned about the wearer's undergarment during ordinary use. In particular configurations, the overall area of the panel fastener 44 on an individual wing-panel 42 can be arranged such that such fastener area will be approximately centered on the longitudinal centerline 52 of the article when the wing-panel is in its in-use position.

In the construction of the article 20, the various components may be assembled and held together with any operative securement mechanism or system. For example, the desired attachments or securements can include adhesive bonds, cohesive bonds, thermal bonds, ultrasonic bonds, pins, snaps, staples, rivets, stitches, welds, zippers, or the like, as well as combinations thereof.

By incorporating its various aspects, features and configurations, alone or in desired combinations, the article of the invention can more effectively maintain a desired positioning in a wearer's undergarment. The article can be less susceptible to excessive shifting and can more effectively provide a desired fit on the wearer. As a result, the article can more effectively prevent undesired leakage, and can more effectively provide desired levels of comfort.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A personal care absorbent article having a bodyside surface, a garment-side surface, a longitudinal-direction, a lateral cross-direction, an intermediate portion interposed between a pair of longitudinally opposed end portions, and a pair of longitudinally-extending, laterally-opposed side edge regions, said article comprising
a backsheet;
a liquid-permeable topsheet;
an absorbent body sandwiched between said backsheet and said topsheet;
at least one pair of wing-panels, a first wing-panel attached to a first side edge region in the intermediate portion of said article, and a second wing-panel attached to a second side edge region in the intermediate portion of said article, each wing-panel configured to wrap about an undergarment of a wearer; and
a panel fastener joined to each wing-panel, each panel fastener configured to operatively secure its corresponding wing-panel about the undergarment of the wearer during ordinary use;
wherein
each wing-panel includes a panel base section which is operatively joined to its corresponding side edge region of the article, and a panel free-end region which extends from the panel base section and is configured to operatively wrap around the undergarment of the wearer;
an overall total, wing-panel area in a first lateral-half of the article is bilaterally asymmetric with respect to an overall total, wing-panel area in a second lateral-half of the article; and
each panel fastener is configured to have a fastener end-spacing of not more than a maximum of about 18% of an overall length of the absorbent body.

2. A personal care article as recited in claim 1, wherein each panel fastener is configured to have a fastener end-spacing of not more than a maximum of about 4 cm.

3. A personal care article as recited in claim 1, wherein the at least one pair of wing-panels are shaped to be longitudinally asymmetric with respect to each other, a first free-end region of the first wing-panel extending toward a first end portion of the article, and a second free-end region of the second wing-panel extending toward a longitudinally opposite, second end portion of the article.

4. A personal care article as recited in claim 1, wherein the article has an overall article shape that is bilaterally asymmetric.

5. A personal care article as recited in claim 1, further including a garment-attachment mechanism operatively joined to the garment-side surface of the article.

6. A personal care article as recited in claim 5, wherein at least a portion of the garment-attachment mechanism located along a longitudinal centerline of the article.

7. A personal care article as recited in claim 5, wherein each panel fastener and a cooperating portion of the garment-attachment mechanism are configured to operatively sandwich a portion of the undergarment between such panel fastener and its cooperating portion of the garment-attachment mechanism when each wing-panel is in an in-use position.

8. A personal care article as recited in claim 1, wherein each wing-panel has a longitudinally-extending base-length which is at least about 25% of the overall length of the absorbent body.

9. A personal care article as recited in claim 8, wherein each wing-panel has a longitudinally-extending base-length which is not more than a maximum of about 42% of the overall length of the absorbent body.

10. A personal care article as recited in claim 8 wherein the article includes first and second, longitudinally opposed, article end-edges; and the panel base section of each wing panel is longitudinally spaced from each article end-edge by a base spacing-distance which is not less than a minimum of about 25% of an overall length of the absorbent body.

11. A personal care article as recited in claim 1, wherein said panel base section has a longitudinally-extending base-length which is at least a minimum of about 6 cm.

12. A personal care article as recited in claim 11, wherein said panel base section has a longitudinally-extending base-length which is not more than about 10 cm.

13. A personal care article as recited in claim 12, wherein each wing-panel and its corresponding panel fastener are configured to have a fastener angle which is not more than a maximum of about 160 degrees.

14. A personal care article as recited in claim 1, wherein the at least one pair of wing-panels are configured to substantially avoid overlapping each other when the wing-panels are in an in-use position.

15. A personal care article as recited in claim 14, wherein the at least one pair of wing-panels are configured to provide a minimum inter-panel gap between the wing-panels of not less than about 0.5 cm when the wing-panels are in the in-use position.

16. A personal care article as recited in claim 1, wherein each wing-panel includes a separately provided wing-panel member which is operatively attached to at least one of said backsheet and topsheet.

17. A personal care article as recited in claim 1, wherein each wing-panel includes
an integrally-formed side portion of said backsheet which extends laterally past a corresponding, terminal side edge of the absorbent body; and
an integrally-formed side portion of said topsheet which extends laterally past a corresponding, terminal side edge of the absorbent body.

18. A personal care article as recited in claim 1, wherein each wing-panel includes a panel composite, said panel composite including
an integrally-formed side portion of said backsheet which extends laterally past a corresponding, terminal side edge of the absorbent body, and
an integrally-formed cooperating, side portion of said topsheet which extends laterally past said corresponding, terminal side edge of the absorbent body, and is positioned in facing relation with said integrally-formed side portion of the backsheet.

19. A personal care article as recited in claim 1, wherein at least about 80% of an overall area of a first panel fastener located in a first lateral-half of the article is located in a first longitudinal half-length of the article; and at least about 80% of an overall area of a second panel fastener located in a second lateral-half of the article is located in a second longitudinal half-length of the article.

20. A personal care article as recited in claim 1, wherein at least about 85% of an overall total wing-panel area positioned in a first lateral-half of the article is located in a first longitudinal half-length of the article; and at least about 85% of an overall total wing-panel area positioned in a second lateral-half of the article is located in a second longitudinal half-length of the article.
